(19) United States
(12) Patent Application Publication
(10) Pub. No.: US 2024/0226039 A1
(43) Pub. Date: Jul. 11, 2024

(54) IMMUNE CHECKPOINT INHIBITOR AND HDAC INHIBITOR COMBINATION THERAPY STRATEGY

(71) Applicant: H. Lee Moffitt Cancer Center and Research Institute, Inc., Tampa, FL (US)

(72) Inventors: Amer BEG, Tampa, FL (US); Xiaoqing YU, Tampa, FL (US)

(21) Appl. No.: 18/563,581

(22) PCT Filed: May 24, 2022
(86) PCT No.: PCT/US2022/072527

§ 371 Date: Nov. 22, 2023

Related U.S. Application Data
(60) Provisional application No. 63/192,313, filed on May 24, 2021.

(51) Int. Cl. A61K 31/167; A61K 39/395; A61P 35/00; C12Q 1/6886; A61K 39/00

(52) U.S. Cl. CPC A61K 31/167; A61K 39/3955; A61P 35/00; C12Q 1/6886; A61K 2039/505; C12Q 2600/106; C12Q 2600/158

(57) ABSTRACT

Disclosed herein is a method for treating a solid tumor in a subject that involves assaying a sample from the subject for expression of two or more Histone deacetylases (HDACs); determining a response score from the expression of the two or more HDACs, wherein the response score predicts whether the subject will respond to combination immunotherapy and HDAC inhibitor therapy; and administering to the subject a therapeutically effective amount of a combination of immunotherapy and an HDAC inhibitor.

### CROSS-REFERENCE TO RELATED APPLICATIONS

This application claims benefit of U.S. Provisional Application No. 63/192,313, filed May 24, 2021, which is hereby incorporated herein by reference in its entirety.

### STATEMENT REGARDING FEDERALLY SPONSORED RESEARCH OR DEVELOPMENT

This invention was made with Government Support under Grant No. W81XWH-18-1-0409 awarded by the Department of Defense, and Grant No. CA076292 awarded by the National Institutes of Health. The Government has certain rights in the invention.

### BACKGROUND

Anti-PD-L1/PD-1 immune checkpoint inhibitors (ICI) can lead to durable tumor regression in up to 20% of patients with advanced non-small cell lung cancer (NSCLC). However, 80% of patients do not significantly benefit from ICI leaving a large unmet medical need. In many other cancer types, such as breast, colon and pancreatic, the ICI response rate is even lower.

### SUMMARY

Disclosed herein is a method for treating a solid tumor in a subject that involves assaying a sample from the subject for expression of two or more Histone deacetylases (HDACs); determining a response score from the expression of the two or more HDACs, wherein the response score predicts whether the subject will respond to combination immunotherapy and HDAC inhibitor therapy; and administering to the subject a therapeutically effective amount of a combination of immunotherapy and an HDAC inhibitor.

The method can in some embodiments be used with any combination of expression of HDAC1, HDAC 2, HDAC 3, HDAC 4, HDAC 5, HDAC 6, HDAC 7, HDAC 8, HDAC 9, HDAC 10, and HDAC 11. In some embodiments, step (a) comprises assaying the sample for expression of 1, 2, 3, 4, or 5 of HDAC1, HDAC2, HDAC3, HDAC4, and HDAC5.

In some embodiments, step (b) involves determining the ratio of a first set of HDACs to a second set of HDACs, wherein a low ratio is an indication that the subject will respond to combination checkpoint inhibitor and HDACi therapy. In some embodiments, the first set can include HDAC1, HDAC 2, and/or HDAC 4. The second set can include HDAC3 and/or HDAC 5.

In some embodiments, a “low ratio” includes any ratio below 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, or 1.6.

In some embodiments, ratio of combined expression of HDAC 1, 2, and/or 4 to the combined expression of HDAC 3 and/or 5 for each sample can be determined to assess response to combination checkpoint inhibitor and HDACi therapy. For example, in some embodiments, ratio of combined expression of HDAC1, HDAC 2, and HDAC 4 to the combined expression of HDAC3 and HDAC5 for each sample is determined to assess response to combination checkpoint inhibitor and HDACi therapy. In some embodiments, ratio of combined expression of HDAC1 and HDAC2 to the combined expression of HDAC3 and HDAC5 for each sample is determined to assess response to combination checkpoint inhibitor and HDACi therapy. In some embodiments, ratio of expression of HDAC1 to the expression of HDAC3 for each sample is determined to assess response to combination checkpoint inhibitor and HDACi therapy. In some embodiments, ratio of expression of HDAC1 to the expression of HDAC5 for each sample is determined to assess response to combination checkpoint inhibitor and HDACi therapy. In some embodiments, ratio of expression of HDAC2 to the expression of HDAC3 for each sample is determined to assess response to combination checkpoint inhibitor and HDACi therapy. In some embodiments, ratio of expression of HDAC2 to the expression of HDAC5 for each sample is determined to assess response to combination checkpoint inhibitor and HDACi therapy. In some embodiments, ratio of expression of HDAC4 to the expression of HDAC3 for each sample is determined to assess response to combination checkpoint inhibitor and HDACi therapy. In some embodiments, ratio of expression of HDAC4 to the expression of HDAC5 for each sample is determined to assess response to combination checkpoint inhibitor and HDACi therapy.

In some embodiments, step (a) further comprises assaying the sample for expression of a housekeeping gene, and wherein step (b) comprises normalizing expression of the two or more HDACs. For example, in some embodiments, the expression is normalized using β-actin or ribosomal RNA expression.

In some embodiments, the immunotherapy is a checkpoint inhibitor, vaccine, an oncolytic virus, a monoclonal antibody, a cell-based immunotherapy such as TIL or CAR-T, or a radiopharmaceutical.

In some embodiments, the checkpoint inhibitor is an anti-PD-1 antibody, anti-PD-L1 antibody, anti-CTLA-4 antibody, or a combination thereof. For example, the checkpoint inhibitor can be pembrolizumab, nivolumab, cemiplimab, atezolizumab, avelumab, or durvalumab.

In some embodiments, the HDAC inhibitor is a pan HDAC inhibitor. In some embodiments, the HDAC inhibitor is a class I HDAC inhibitor. In some embodiments, the HDAC inhibitor is a class IIA HDAC inhibitor. In some embodiments, the HDAC inhibitor is a class III HDAC inhibitor. In some embodiments, the HDAC inhibitor is a class IV HDAC inhibitor. For example, in some embodiments, the HDAC inhibitor is vorinostat, entinostat, panobinostat, romidepsin, belinostat, captinostat, mocetinostat, givinostat, psinostat, chidamide, or quisininostat. In some embodiments, the HDAC inhibitor is HBI-8000.

The disclosed methods can in some embodiments be used to treat any solid tumor. In some embodiments, the tumor is not treatable or is refractive to immunotherapy. In some embodiments, the tumor is a prostate cancer, breast cancer, ovarian cancer, lung cancer, or colon cancer.

In some embodiments, the method further involves treating the tumor cells with one or more additional therapies, such as a chemotherapy and/or radiation therapy.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a spider plot showing change in target lesions over time.

FIGS. 2A and 2B contain waterfall plots of best response, defined as percent change from baseline sum of target lesion diameters. FIG. 2A shows Arm A (pembrolizumab), and FIG. 2B shows Arm B (pembrolizumab+vorinostat).

FIG. 3 contains bar plots showing the proportion of patients with no (0), low (1) or moderate (2) CD8+ TIL on pre-treatment biopsy, stratified by response.

FIG. 4 shows pre-treatment biopsies from two patients in Arm B with low CD8+ TIL who had a partial response to treatment. H&E (left), and IHC highlighting CD8+ (brown) and CD33+ (red) cells (right). Tumor (T) and stromal (S) regions are marked.

FIG. 5 contains line graphs for Arm A (pembrolizumab) and Arm B (pembrolizumab+vorinostat), showing the change in CD8+ TIL for individual patients.

FIGS. 6A to 6K show relative RNA expression (determined by RNA sequencing) in Arm A pre-treatment, Arm A on-treatment C1D15, Arm B pre-treatment, Arm B on-treatment C1D15 for HDAC1 (FIG. 6A), HDAC2 (FIG. 6B), HDAC3 (FIG. 6C), HDAC4 (FIG. 6D), HDAC5 (FIG. 6E), HDAC6 (FIG. 6F), HDAC7 (FIG. 6G), HDAC8 (FIG. 6H), HDAC9 (FIG. 6I), HDAC10 (FIG. 6J), and HDAC11 (FIG. 6K) in Arm A and Arm B. No observable clear differences were noted between the 4 sample sets.

FIGS. 7A to 7V show relative RNA expression in Arm A and Arm B biopsies separated based on patient response. Progressive disease (PD) and Stable disease (SD) biopsies for each Arm were in one group that did not receive benefit. Partial response (PR) were in a separate group that received benefit from treatment. Expression for HDAC1 (FIGS. 7A, 7B), HDAC2 (FIG. 7C, 7D), HDAC3 (FIG. 7E, 7F), HDAC4 (FIG. 7G, 7H), HDAC5 (FIG. 7I, 7J), HDAC 6 (FIG. 7K, 7L), HDAC 7 (FIG. 7M, 7N), HDAC 8 (FIG. 7O, 7P), HDAC 9 (FIG. 7Q, 7R), HDAC 10 (FIG. 7S, 7T), and HDAC 11 (FIG. 7U, 7V) for Arm A (FIGS. 7A, 7C, 7E, 7G, 7I, 7K, 7M, 7O, 7Q, 7S, 7U) and Arm B (FIGS. 7B, 7D, 7F, 7H, 7J, 7L, 7N, 7P, 7R, 7T, 7V) for PD and SD vs. PR.

FIG. 8 shows combined low expression of both HDAC1 and HDAC2 is associated with response.

FIG. 9 shows samples ranked based on the ratio of log2(HDAC1+HDAC2)/log2(HDAC3+HDAC5) in Arm A and Arm B separately. Samples are colored by best response, and shaped by condition (PRE or On-Treatment).

FIG. 10 shows samples ranked based on the ratio of log2(HDAC1+HDAC2)/log2(HDAC3+HDAC5) in Arm A and Arm B for pre-treatment and on-treatment C1D15. Samples are colored by best response, and shaped by condition (PRE or On-Treatment).

FIG. 11 is a boxplot comparing log2(HDAC1+HDAC2)/log2(HDAC3+HDAC5) values between patients categorized by conditions and best responses, in Arm A and Arm B separately. P-values are shown for Wilcoxon test.

FIG. 12 shows samples ranked based on the ratio of log2(HDAC1)/log2(HDAC5) in Arm A and Arm B separately. Samples are colored by best response and shaped by condition (PRE or Treatment).

FIG. 13 is a boxplot comparing log2(HDAC1)/log2(HDAC5) values between patients categorized by conditions and best responses, in Arm A and Arm B separately. P-values are shown for Wilcoxon test. Samples are colored by best response and shaped by condition (PRE or Treatment).

FIG. 14 shows samples ranked based on the ratio of log2(HDAC1)/log2(HDAC3) in Arm A and Arm B separately. Samples are colored by best response and shaped by condition (PRE or Treatment). Samples are colored by best response and shaped by condition (PRE or Treatment).

FIG. 15 is a boxplot comparing log2(HDAC1)/log2(HDAC3) values between patients categorized by conditions and best response, in Arm A and Arm B separately. P-values are shown for Wilcoxon test.

FIG. 16 shows samples ranked based on the ratio of log2(HDAC2)/log2(HDAC3) in Arm A and Arm B separately. Samples are colored by best response and shaped by condition (PRE or Treatment).

FIG. 17 is a boxplot comparing log2(HDAC2)/log2(HDAC3) values between patients categorized by conditions and best response, in Arm A and Arm B separately. P-values are shown for Wilcoxon test.

FIG. 18 shows samples ranked based on the ratio of log2(HDAC1+HDAC2+HDAC4)/log2(HDAC3+HDAC5) in Arm A and Arm B separately for pre-treatment and on-treatment C1D15.

FIG. 19 is a boxplot comparing log2(HDAC1+HDAC2+HDAC4)/log2(HDAC3+HDAC5) values between patients categorized by conditions and best responses, in Arm A and Arm B separately. P-values are shown for Wilcoxon test.

FIG. 20 shows samples ranked based on the ratio of log2(HDAC2)/log2(HDAC3) in Arm A and Arm B for pre-treatment and on-treatment C1D15.

FIG. 21 is a boxplot comparing log2(HDAC2)/log2(HDAC3) values between patients categorized by conditions and best responses, in Arm A and Arm B separately. P-values are shown for Wilcoxon test.

FIG. 22 shows samples ranked based on the ratio of log2(HDAC1+HDAC2)/log2(HDAC3+HDAC5) from a study of PD-(L)1 refractory NSCLC patients (Gray, J. E., et al. Clin Cancer Res. 2019 25(22):6623-6632). Benefit was defined as partial response or stable disease greater than 24 weeks. No-Benefit is progressive disease or stable disease less than 24 weeks.

FIG. 23 shows association of progression free survival (PFS) with HDAC ratios log2(HDAC1+HDAC2)/log2(HDAC3+HDAC5) in study of PD-(L) 1 refractory NSCLC patients (Gray, J. E., et al. Clin Cancer Res. 2019 25(22):6623-6632). Low HDAC ratio is associated with higher PFS but only in patients with treatment benefit. Benefit defined as partial response or stable disease greater than 24 weeks.

FIG. 24 shows samples ranked based on the ratio of log2(HDAC1+HDAC2)/log2(HDAC3+HDAC5) from a study of PD-(L)1 refractory melanoma patients. Patient response is indicated as well as time of biopsy collection.

FIG. 25 is a boxplot comparing the ratio of log2(HDAC1+HDAC2)/log2(HDAC3+HDAC5) values from a study of PD-(L)1 refractory melanoma patients. Patient response is indicated, biopsies collected before treatment were used. P-values are shown for Wilcoxon test.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood (e.g. PBMC in blood), urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### HDAC Expression

The disclosed methods can include analyzing a sample for levels of one or more HDACs. The sample can in some cases be analyzed for mRNA or protein expression. The levels can be identified (determined) using techniques that include, for example, DNA microarray, high-density oligonucleotide microarray, whole-genome RNA expression array, NanoString panel (e.g. a customized panel for HDAC expression), RNA-sequencing, peptide microarray, enzyme-linked immunosorbent assay (ELISA), genome sequencing, de novo sequencing, 454 sequencing, pyrosequencing, Helicos True Single Molecule Sequencing, SOLID™ sequencing, SOLEXA sequencing, nanosequencing, chemical-sensitive field effect transistor (chemFET) array sequencing, polony sequencing, copy number variation (CNV) analysis sequencing, small nucleotide polymorphism (SNP) analysis, immunohistochemistry (IHC), immunocytochemistry (ICC), mass spectrometry, tandem mass spectrometry, matrix-assisted laser desorption ionization time of flight mass spectrometry (MALDI-TOF MS), in-situ hybridization, fluorescent in-situ hybridization (FISH), chromogenic in-situ hybridization (CISH), silver in situ hybridization (SISH), polymerase chain reaction (PCR), digital PCR (dPCR), reverse transcription PCR, quantitative PCR (Q-PCR), single marker qPCR, real-time PCR, nCounter Analysis (Nanostring technology), Western blotting, Southern blotting, SDS-PAGE, gel electrophoresis, and Northern blotting.

### HDAC Inhibitor

Any suitable HDAC inhibitor can be used in the methods described herein. Exemplary HDAC inhibitors include, but are not limited to, hydroxamates (e.g., TSA, vorinostat, M-Carboxycinnamic acid bishydroxamate (CBHA) and derivatives thereof (e.g., LAQ-824, belinostat (PDX-101), and Panobinostat (LBH-589)), ITF2357 (Italfarmaco SpA), and PC1-24781), cyclic peptides (e.g., depsipeptide (FK-228), apicidin, and the cyclic hydroxamic acid-containing peptide group of molecules), aliphatic acids (valproic acid, phenyl butyrate, butyrate, and pivaloyloxymethyl butyrate (AN-9)), and benzamides or derivatives thereof (5 NOX-275 (MS-275), MGCD0103, and entinostat) (Dokmanovic, Mol. Cancer Res., 5: 981 (2007)). In one embodiment, the HDAC inhibitor is selected from the group consisting of apcidin, belinostat, entinostat, mocetinostat, panobinostat, abexinostat, PC1-334051, romidepsin, vorinostat, trichostatin A, and valproic acid (West et al, J. Clin. Invest. 124(1): 30-39 (2014)).

In some embodiments, the HDAC inhibitor is a HDAC inhibitor is a class I HDAC inhibitor. Exemplary, class I HDAC inhibitors include apcidin, belinostat, entinostat, mocetinostat, panobinostat, abexinostat, romidepsin, vorinostat, trichostatin A, and valproic acid. In one embodiment, the HDAC inhibitor is vorinostat or entinostat.

In some embodiments, the combination of immunotherapy and a HDAC inhibitor reduces or inhibits growth of cancer cells (e.g., prostate cancer cells, breast cancer cells, lung cancer cells, or colon cancer cells). The term “growth,” as used herein, encompasses any aspect of the growth, proliferation, and progression of cancer cells, including, for example, cell division (i.e., mitosis), cell growth (e.g. increase in cell size), an increase in genetic material (e.g., prior to cell division), and metastasis. Reduction, inhibition, or suppression of cancer cell growth includes, but is not limited to, inhibition of cancer cell growth as compared to the growth of untreated or mock treated cells, inhibition of proliferation, inhibition of metastases, sensitization to immune-mediated killing (e.g., T-cell-mediated lysis), induction of cancer cell senescence, induction of cancer cell death, and reduction of tumor size.

### Immunotherapy

The term “immunotherapy,” as used herein refers to the treatment of a disease by inducing, enhancing, or suppressing an immune response. Immunotherapies designed to elicit or enhance an immune response are referred to as activation immunotherapies, while immunotherapies designed to suppress an immune response are referred to suppression immunotherapies. Types of immunotherapies include, but are not limited to, checkpoint inhibitors, immunomodulators, cell-based immunotherapies, monoclonal antibodies, radiopharmaceuticals, and vaccines. Immunotherapy strategies for cancer are described in, for example, Waldmann, T. A., Nature Medicine, 9: 269-277 (2003).

Immunomodulators can be recombinant, synthetic, or natural substances that include, but are not limited to, cytokines (e.g., TNF-α, IL-6, GM-CSF, IL-2, and interferons), co-stimulatory molecules (e.g., B7-1 and B7-2), chemokines (e.g., CCL3, CCL26, CXCL7), glucans, and oligodeoxynucleotides.

Cell-based immunotherapies typically involve removal of immune cells (e.g., cytotoxic T-cells, natural killer cells, or antigen presenting cells (APCs)) from a subject, modification (e.g., activation) of immune cells, and return of the modified immune cells to the patient. In the context of the inventive method, the cell-based immunotherapy desirably is Sipuleucel-T (PROVENGE™), which is an autologous active cellular immunotherapy used in the treatment of asymptomatic or minimally symptomatic CRPC (Plosker, G. L., Drugs, 71(1): 101-108 (2011); and Kantoff et al., New Engl. J. Med., 363: 411-422 (2010)).

Several monoclonal antibodies have been approved for the treatment of cancer, including naked antibodies and antibody-drug conjugates based on human, humanized, or chimeric antibodies (Scott et al., Nat Rev Cancer, 12(4): 278-87 (2012); Harding et al., MAbs, 2(3): 256-65 (2010); and Weiner et al., Nature Rev. Immunol., 10(5): 317-327 (2010)). In one embodiment, the inventive method comprises treating the prostate cancer cells with any suitable monoclonal antibody known in the art. Such monoclonal antibodies include, for example, ipilumimab (YERVOY™), which is a fully human antibody that binds to CTLA-4 and is indicated for the treatment of melanoma. Antibodies that target the interaction of programmed death receptor-1 (PD-1) with its ligands PD-L1 and PD-L2, also can be used in the invention (see, e.g., Weber, Semin. Oncol., 37(5): 430-4309 (2010); and Tang et al., Current Oncology Reports, 15(2): 98-104 (2013)). Antibodies that inhibit PD-1 signaling include, for example nivolumab (also known as BMS-936558 or MDX1106; see, e.g., ClinicalTrials.gov Identifier NCT00730639), sipuleucel-T CT-011, pembrolizumab, atezolizumab, and MK-3575 (see, e.g., Patnaik et al., 2012 American Society of Clinical Oncology (ASCO) Annual Meeting, Abstract #2512). Monoclonal antibodies that specifically target prostate cancer are under development and also can be used in the invention (see, e.g., Jakobovits, A., Handb. Exp. Pharmacol., 181: 237-56 (2008); and Ross et al., Cancer Metastasis Rev., 24(4): 521-37 (2005)). Monoclonal antibodies suitable for treatment of breast cancer include, for example, trastuzumab (HERCEPTIN™), pertuzumab (PERJETA™), and the antibody-drug conjugate ado-trastuzumab emtansine (KADCYLA™).

### Treatment

When a HDAC inhibitor is administered with one or more immunotherapeutic agents, the HDAC inhibitor and one or more immunotherapeutic agents can be co-administered to the mammal. By “co-administering” is meant administering one or more immunotherapeutic agents and the HDAC inhibitor sufficiently close in time such that the HDAC inhibitor can enhance the effect of the one or more immunotherapeutic agents. In this regard, the HDAC inhibitor can be administered first and the one or more immunotherapeutic agents can be administered second, or vice versa. Alternatively, the HDAC inhibitor and the one or more immunotherapeutic agents can be administered simultaneously.

The combination of the HDAC inhibitor and immunotherapy can be administered to a subject by various routes including, but not limited to, oral, subcutaneous, intramuscular, intradermal, intraperitoneal, intravenous, and intratumoral. When multiple administrations are given, the administrations can be at one or more sites in a subject.

Administration of the combination can be “prophylactic” or “therapeutic.” When provided prophylactically, the combination is provided in advance of tumor formation to allow the host's immune system to fight against a tumor that the host is susceptible of developing. For example, hosts with hereditary cancer susceptibility are a preferred group of patients treated with such prophylactic immunization. The prophylactic administration of a HDAC inhibitor or a composition thereof prevents, ameliorates, or delays cancer. When provided therapeutically, the combination is provided at or after the diagnosis of cancer. When the host has already been diagnosed with cancer (e.g., metastatic cancer), the combination can be administered in conjunction with other therapeutic treatments such as chemotherapy or radiation.

The following formulations for oral, aerosol, parenteral (e.g., subcutaneous, intravenous, intraarterial, intramuscular, intradermal, interperitoneal, and intrathecal), rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The combination of the HDAC inhibitor and immunotherapy can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The HDAC inhibitor, immunotherapeutic agent, and/or compositions thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets.

The HDAC inhibitor, immunotherapeutic agent, and/or compositions thereof can be administered as an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topical formulations, including those that are useful for transdermal drug release, are well known to those of skill in the art and are suitable in the context of the invention for application to skin.

The HDAC inhibitor, immunotherapeutic agent, and/or compositions thereof can be administered as a suppository by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Methods for preparing administrable (e.g., parenterally administrable) HDAC inhibitors, immunotherapeutic agents, and/or compositions thereof are known or apparent to those skilled in the art and are described in more detail in, for example, Remington's Pharmaceutical Science (17th ed., Mack Publishing Company, Easton, Pa., 1985).

In addition to the aforedescribed pharmaceutical compositions, the HDAC inhibitor, immunotherapeutic agent, and/or compositions thereof can be formulated as inclusion complexes, such as cyclodextrin inclusion complexes, or liposomes. Liposomes can serve to target the HDAC inhibitor, immunotherapeutic agent, and/or compositions thereof to a particular tissue. Liposomes also can be used to increase the half-life of the HDAC inhibitor, immunotherapeutic agent, and/or compositions thereof. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

### EMBODIMENTS

Embodiment 1. A method for treating a solid tumor in a subject, comprising
(a) assaying a sample from the subject for expression of two or more Histone deacetylases (HDACs);
(b) determining a predicted response score from the expression of the two or more HDACs, wherein the response score predicts that the subject will respond and/or benefit from combination immunotherapy and HDAC inhibitor therapy; and
(c) administering to the subject a therapeutically effective amount of a combination of immunotherapy and an HDAC inhibitor.

Embodiment 2. The method of embodiment 1, wherein step (a) comprises assaying the sample for any combination of two or more of HDAC1, HDAC2, HDAC3, HDAC4, and HDAC5.

Embodiment 3. The method of embodiment 2, wherein step (a) comprises assaying the sample for HDAC1, HDAC2, HDAC3, HDAC4, and HDAC5.

Embodiment 4. The method of embodiment 2, wherein step (a) comprises assaying the sample for HDAC1, HDAC2, HDAC3, and HDAC5.

Embodiment 5. The method of any one of embodiments 1 to 4, wherein step (b) comprises determining the ratio of a first set of HDACs to a second set of HDACs, wherein a ratio at or below 1.4 is an indication that the subject will respond to combination checkpoint inhibitor and HDACi therapy.

Embodiment 6. The method of embodiment 5, wherein the first set comprises HDAC1, HDAC2, and/or HDAC4 and the second set comprises HDAC3 and/or HDAC5.

Embodiment 7. The method of embodiment 6, wherein the first set comprises HDAC1 and HDAC2 and the second set comprises HDAC3 and HDAC5.

Embodiment 8. The method of any one of embodiments 1 to 7, wherein step (a) further comprises assaying the sample for expression of a housekeeping gene, and wherein step (b) comprises normalizing expression of the two or more HDACs.

Embodiment 9. The method of any one of embodiments 1 to 8, wherein the immunotherapy is a checkpoint inhibitor, vaccine, a monoclonal antibody, an oncolytic virus, a cell-based immunotherapy, or a radiopharmaceutical.

Embodiment 10. The method of embodiment 9, wherein the checkpoint inhibitor comprises an anti-PD-1 antibody, anti-PD-L1 antibody, anti-CTLA-4 antibody, or a combination thereof.

Embodiment 11. The method of embodiment 10, wherein the checkpoint inhibitor comprises pembrolizumab, nivolumab, cemiplimab, atezolizumab, avelumab, or durvalumab.

Embodiment 12. The method of any one of embodiments 1 to 11, wherein the HDAC inhibitor is a class I HDAC inhibitor.

Embodiment 13. The method of embodiment 12, wherein the HDAC inhibitor is vorinostat, entinostat, romidepsin, or panabinostat.

Embodiment 14. The method of embodiment 12, wherein the HDAC inhibitor is HBI-8000.

Embodiment 15. The method of any one of embodiments 1 to 14, wherein the tumor is a melanoma.

Embodiment 16. The method of embodiment 15, wherein the subject is refractory to immunotherapy.

Embodiment 17. The method of any one of embodiments 1 to 14, wherein the tumor is a non-small cell lung cancer (NSCLC)

Embodiment 18. The method of embodiment 17, wherein the subject is immunotherapy naïve.

Embodiment 19. The method of any one of embodiments 1 to 18, further comprising treating the tumor cells with one or more additional therapeutic agents.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Phase II Randomized Trial of First-Line Pembrolizumab and Vorinostat in Patients With Metastatic NSCLC (mNSCLC)

The oral histone deacetylase inhibitor (HDACi) vorinostat enhances tumor immunogenicity and may augment response to ICI through several mechanisms, including induced expression of T cell chemokines such as Cxcl9 and Cxcl10 and increased T cell trafficking into tumors. The combination of pembrolizumab with vorinostat in mNSCLC was well tolerated with signals of activity in ICI-pretreated patients. A randomized, phase II trial in the first-line setting was initiated with the primary objective to determine if the combination had superior ORR compared to pembrolizumab monotherapy.

### Methods

Key inclusion criteria: Histologically confirmed mNSCLCm Treatment naïve, and PD-L1 expression≥1% are eligible. Key exclusion criteria: Untreated, progressive, or symptomatic brain metastases, Active uncontrolled autoimmune disorders, and Systemic steroid use equivalent to >10 mg prednisone. Patients were randomized open-label 1:1 to receive [Arm A] pembrolizumab 200 mg IV q3 wks, or [Arm B] pembrolizumab 200 mg IV q3 wks+vorinostat 400 mg PO daily. The primary endpoint was overall response rate (ORR). Additional endpoints included correlative analyses, DOR, PFS and OS. The trial used a Simon Mini-Max two-stage design with interim analysis planned after the first stage when 23 patients were enrolled in each arm.

Tissue was obtained from core biopsy both prior to starting treatment, and between pembrolizumab cycle 1 days 15-21. IHC was performed to determine presence of CD8+ T cells and CD33+ myeloid cells. Biopsies were scored using a 0-3 scale separately in the tumor stroma and tumor beds.

### Results

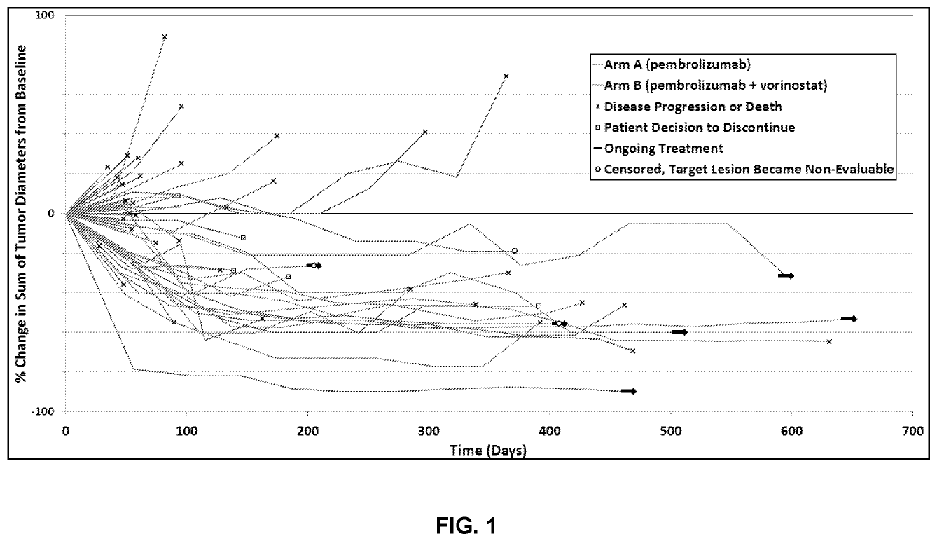

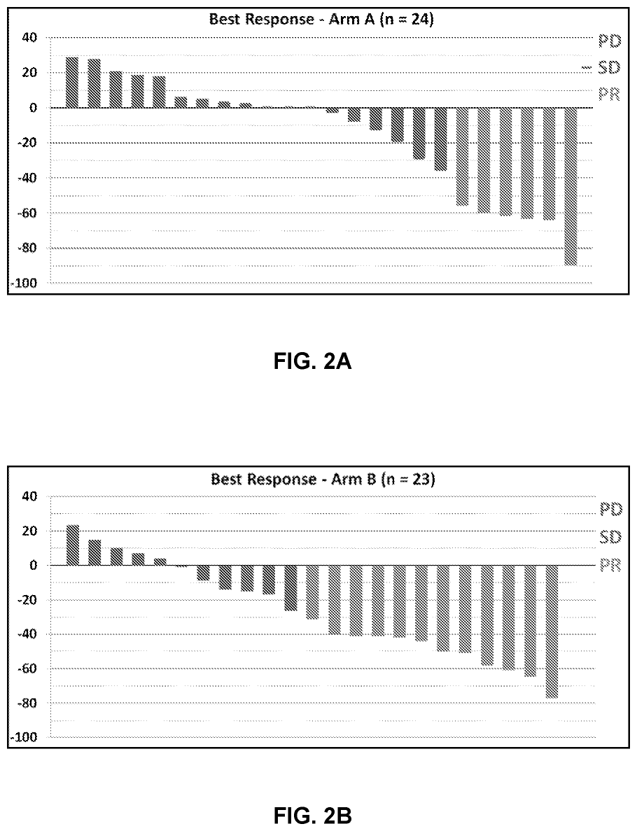

49 patients were enrolled, with 47 patients evaluable for response (24 in Arm A and 23 in Arm B). Baseline characteristics are summarized in Table 1. Adverse events are summarized in Table 2, and immune-mediated AEs in Table 3. 2 patients in Arm A (8%) and 2 patients in Arm B (8%) had treatment discontinued due to toxicity. Dose reductions of vorinostat (non-mandated) were made in 12 (52%) of the patients in Arm B (after a median of 3 cycles, range 1-14) under the discretion of the treating physician or study PI. All TRAEs during dose reduction were grade 1 or 2. Response data are summarized in Table 4, FIGS. 1, 2A, and 2B.

TABLE 1
Baseline Patient Characteristics *NGS not available for all patients

| | | Arm A | Arm B |
|---|---|---|---|
| N | | 24 | 23 |
| Age, Median (Range) | | 69 (47-86) | 69 (55-87) |
| ECOG PS | 0 | 1 (4%) | 4 (17%) |
| | 1 | 23 (96%) | 19 (83%) |
| Gender | F | 11 (46%) | 12 (52%) |
| | M | 13 (54%) | 11 (48%) |
| Smoking Status | Never | 0 (0%) | 5 (22%) |
| | Former | 16 (73%) | 17 (74%) |
| | Current | 6 (27%) | 1 (4%) |
| PD-L1 TPS | 1-49% | 11 (46%) | 10 (43%) |
| | >50% | 13 (54%) | 13 (57%) |
| Histology | Adenocarcinoma | 19 (79%) | 20 (87%) |
| | Squamous | 5 (21%) | 3 (13%) |
| Mutation Status* | KRAS | 6 (25%) | 7 (30%) |
| | ROS1 | 0 (0%) | 1 (4%) |
| | BRAF (non-V600) | 1 (4%) | 1 (4%) |
| | TP53 | 5 (21%) | 2 (9%) |
| | STK11 | 0 (0%) | 1 (4%) |
| | EGFR | 0 (0%) | 0 (0%) |
| | ALK | 0 (0%) | 0 (0%) |

TABLE 2
AEs attributed as related to study treatment, occurring in ≥5%
of patients (or Grade 3/4). There were no Grade 5 AEs.

| | Arm A (n = 24) | | | | |
|---|---|---|---|---|---|
| Adverse Event Detail | Gr 1 | Gr 2 | Gr 3 | Gr 4 | Total |
| Anorexia | 1 (4%) | 0 | 0 | 0 | 1 (4%) |
| Fatigue | 1 (4%) | 1 (4%) | 0 | 0 | 2 (8%) |
| Creatinine increased | 0 | 0 | 0 | 0 | 0 |
| Nausea | 0 | 0 | 0 | 0 | 0 |
| Diarrhea | | 1 (4%) | 0 | 0 | 3 (13%) |
| Dysgeusia | 0 | 0 | 0 | 0 | 0 |
| Pneumonitis | 0 | 0 | 1 (4%) | 0 | 1 (4%) |
| Alopecia | 0 | 0 | 0 | 0 | 0 |
| Anemia | 0 | 0 | 0 | 0 | 0 |
| Vomiting | 0 | 0 | 0 | 0 | 0 |
| Pain | 0 | 0 | 0 | 0 | 0 |
| Arthralgia | 0 | 0 | 0 | 0 | 0 |
| Platelet count decreased | 0 | 0 | 0 | 0 | 0 |
| Rash maculo-papular | 0 | 0 | 1 (4%) | 0 | 1 (4%) |
| Pruritis | | 0 | 0 | 0 | |
| ALT increased | 0 | 0 | 1 (4%) | 0 | 1 (4%) |
| AST increased | 0 | 0 | 1 (4%) | 0 | 1 (4%) |
| Hypothyroidism | 0 | 1 (4%) | 0 | 0 | 1 (4%) |
| Thromboembolic event | 0 | 0 | 0 | 0 | 0 |
| Pericardial effusion/ tamponade | 0 | 0 | 0 | 0 | 0 |
| Heart failure | 0 | 0 | 0 | 0 | 0 |
| Alkaline phosphatase increased | 0 | 0 | 0 | 0 | 0 |
| Hyponatremia | 0 | 0 | 1 (4%) | 0 | 1 (4%) |
| Chest pain - cardiac | 0 | 0 | 1 (4%) | 0 | 1 (4%) |
| QT prolongation | 0 | 0 | 0 | 0 | 0 |
| Total | 8 | 6 | 6 | 0 | 20 |

Arm B (n = 23)

| Adverse Event Detail | Gr 1 | Gr 2 | Gr 3 | Gr 4 | Total |
|---|---|---|---|---|---|
| Anorexia | 7 (30%) | 3 (13%) | 0 | 0 | 0 |
| Fatigue | 4 (17%) | 6 (26%) | 0 | 0 | 0 |
| Creatinine increased | 6 (26%) | 2 (9%) | 0 | 0 | 0 |
| Nausea | 2 (9%) | 5 (22%) | 1 (4%) | 0 | 0 |
| Diarrhea | 5 (22%) | 2 (9%) | 0 | 0 | 0 |
| Dysgeusia | 4 (17%) | 3 (13%) | 0 | 0 | 0 |
| Pneumonitis | 0 | 5 (22%) | 1 (4%) | 0 | 0 |
| Alopecia | 4 (17%) | 0 | 0 | 0 | 0 |
| Anemia | 1 (4%) | 2 (9%) | 1 (4%) | 0 | 0 |
| Vomiting | 3 (10%) | 1 (4%) | 0 | 0 | 0 |
| Pain | 3 (15%) | 1 (4%) | 0 | 0 | 0 |
| Arthralgia | 2 (20%) | 1 (4%) | 0 | 0 | 0 |
| Platelet count decreased | 2 (10%) | 1 (4%) | 0 | 0 | 0 |
| Rash maculo-papular | 3 (10%) | 0 | 0 | 0 | 0 |
| Pruritis | 1 (15%) | 0 | 0 | 0 | 0 |
| ALT increased | 1 (4%) | 1 (4%) | 0 | 0 | 0 |
| AST increased | 2 (5%) | 0 | 0 | 0 | 0 |
| Hypothyroidism | 0 | 2 (9%) | 0 | 0 | 0 |
| Thromboembolic event | 0 | 1 (4%) | 1 (4%) | 0 | 1 (4%) |
| Pericardial effusion/ tamponade | 0 | 0 | 0 | 1 (4%) | 0 |
| Heart failure | 0 | 0 | 1 (4%) | 0 | 0 |
| Alkaline phosphatase increased | 0 | 0 | 0 | 0 | 0 |
| Hyponatremia | 0 | 0 | 0 | 0 | 0 |
| Chest pain - cardiac | 0 | 0 | 1 (4%) | 0 | 0 |
| QT prolongation | 0 | 0 | 1 (4%) | 0 | 0 |
| Total | 68 | 45 | 7 | 1 | 1 |

TABLE 3
Immune-mediated adverse events (irAEs)
attributed to study treatment.

| | Arm A (n = 24) | | | | |
|---|---|---|---|---|---|
| Adverse Event Detail | Gr 1 | Gr 2 | Gr 3 | Gr 4 | Total |
| Pneumonitis | 0 | 0 | 1 (4%) | 0 | 1 (4%) |
| Pericardial effusion | 0 | 0 | 0 | 0 | 0 |
| Rash/pruritis/dry skin | 4 (17%) | 0 | 1 (4%) | 0 | 5 (21%) |
| Arthralgia | 0 | 0 | 0 | 0 | 0 |
| Hepatitis | 0 | 0 | 1 (4%) | 0 | 1 (4%) |
| AST increased | 0 | 0 | 0 | 0 | 0 |
| Colitis | 0 | 1 (4%) | 0 | 0 | 1 (4%) |
| Adrenal insufficiency | 0 | 1 (4%) | 0 | 0 | 1 (4%) |
| Hypothyroidism | 0 | 0 | 0 | 0 | 0 |
| Brachial plexopathy | 0 | 0 | 0 | 0 | 0 |
| Pancreatic atrophy | 0 | 0 | 0 | 0 | 0 |
| Total | 4 | 2 | 3 | 0 | 9 |

| | Arm B (n = 23) | | | | |
|---|---|---|---|---|---|
| Adverse Event Detail | Gr 1 | Gr 2 | Gr 3 | Gr 4 | Total |
| Pneumonitis | 0 | 5 (22%) | 1 (4%) | 0 | 6 (26%) |
| Pericardial effusion | 0 | 0 | 0 | 1 (4%) | 1 (4%) |
| Rash/pruritis/dry skin | 5 (22%) | 0 | 0 | 0 | 5 (22%) |
| Arthralgia | 3 (13%) | 3 (13%) | 0 | 0 | 6 (26%) |
| Hepatitis | 0 | 0 | 0 | 0 | 0 |
| AST increased | 1 (4%) | 0 | 0 | 0 | 1 (4%) |
| Colitis | 0 | 0 | 0 | 0 | 0 |
| Adrenal insufficiency | 0 | 0 | 0 | 0 | 0 |
| Hypothyroidism | 0 | 2 | 0 | 0 | 2 |
| Brachial plexopathy | 0 | 1 (4%) | 0 | 0 | 1 (4%) |
| Pancreatic atrophy | 1 (4%) | 0 | 0 | 0 | 1 (4%) |
| Total | | | | | |

TABLE 4
Response summary for Arm A and Arm B.

| Best Response | Arm A (n = 24) | Arm B (n = 23) | p value |
|---|---|---|---|
| Partial Response | 6 (25%) | 12 (52%) | p = 0.026 |
| Stable Disease | 8 (33%) | 9 (39%) | |
| Progressive Disease | 10 (42%) | 2 (9%) | |
| Disease Control Rate | 14 (58%) | 21 (91%) | p = 0.024 |

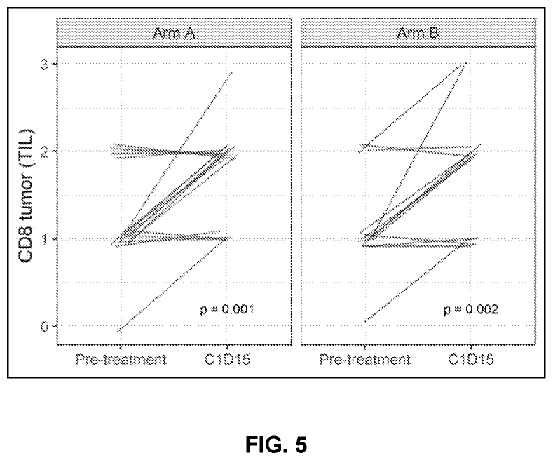

Pre-treatment CD8+ TIL were not significantly different between Arm A and Arm B (p=0.85) with the majority of tumors in both arms having a low tumor bed TIL score of 1 (65% Arm A and 73.7% Arm B). A significant increase from pre-treatment to on-treatment TIL scores was seen in both Arm A (p=0.001) and Arm B (p=0.002) (FIG. 5).

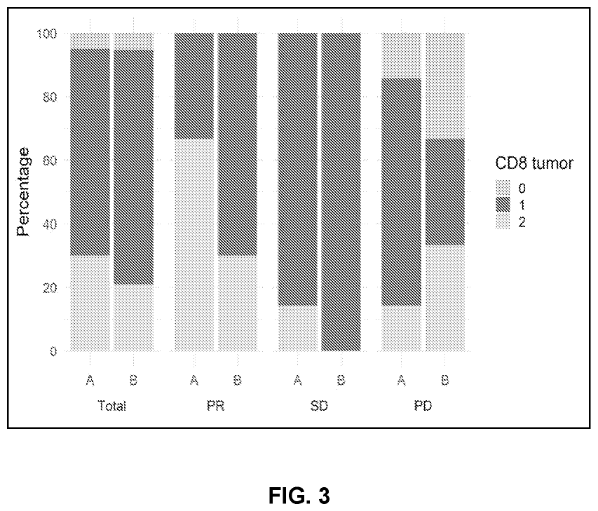

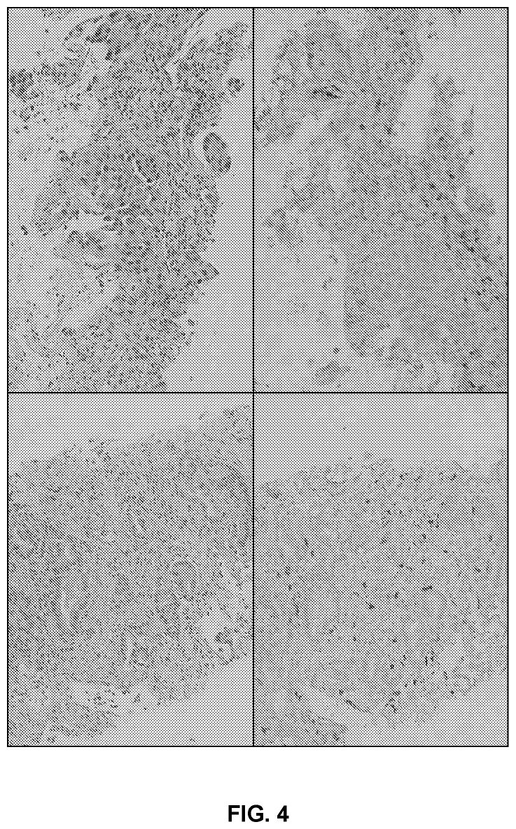

The ORR in Arm B pts with low pre-treatment tumor bed TIL (score=1) was substantially higher (66.7%) than in Arm A (33.3%), suggesting the combination may be especially beneficial against low TIL tumors. (FIGS. 3 and 4).

### Conclusions

The combination arm had a considerably higher ORR and significantly higher disease control rate compared to pembrolizumab monotherapy.

Vorinostat (400 mg PO daily) plus pembrolizumab (200 mg IV q3 week) was relatively well tolerated with primarily grade 1-2 toxicities occurring in <50% of patients. Although there was no observed increase in grade 3-4 irAEs (or discontinuation due to AEs) with the combination therapy, further attention is warranted to a higher rate of grade 1-2 irAEs, including pneumonitis.

Preliminary correlative studies show higher ORR with combination therapy among pts with low baseline CD8+ tumor bed TILs.

### Example 2

### Identification of Biomarkers of Response to the Combination Treatment

Can we define a biomarker in pretreatment or early treatment biopsies that is associated with benefit from the combination treatment? Such a biomarker can allow patient selection specifically for anti-PD-1+HDACi treatment. Biomarker may include a gene-set. In NSCLC, this population may be 25% or higher and may also exist in other cancer types. No clear implemendable biomarker identified in immune genes or in IHC studies performed to date. As vorinostat targets HDACs, studies were conducted to examine whether expression of different HDACs was associated with response to the combination.

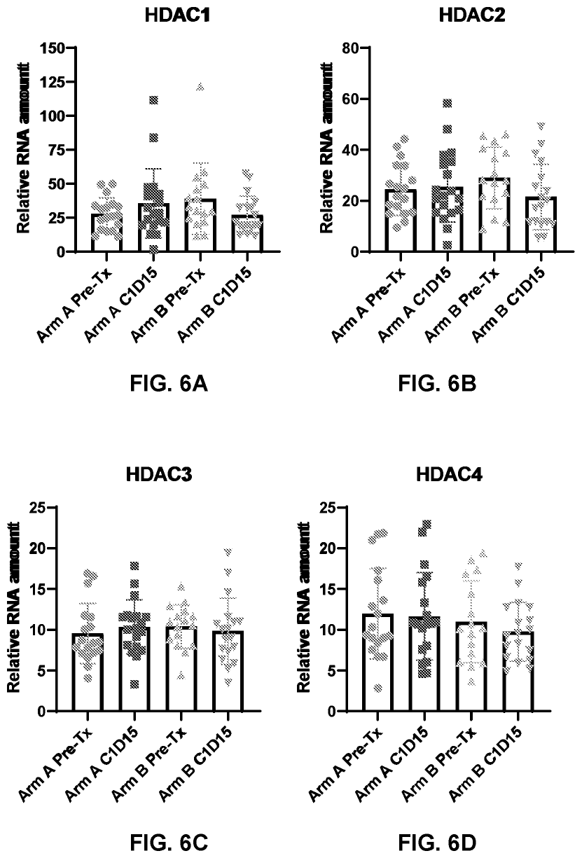

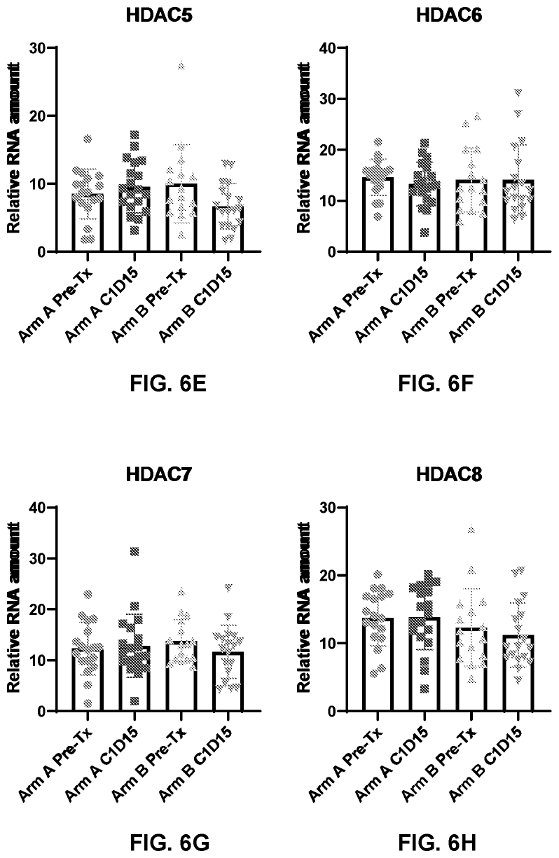

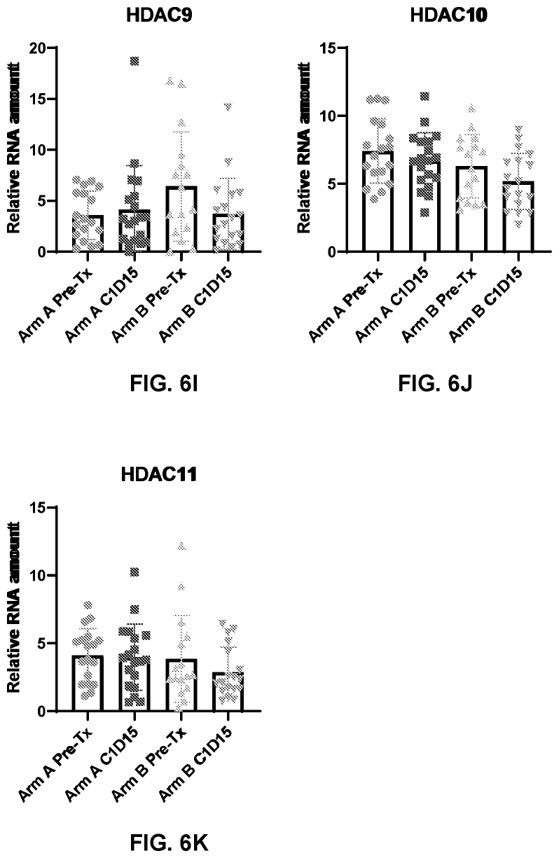

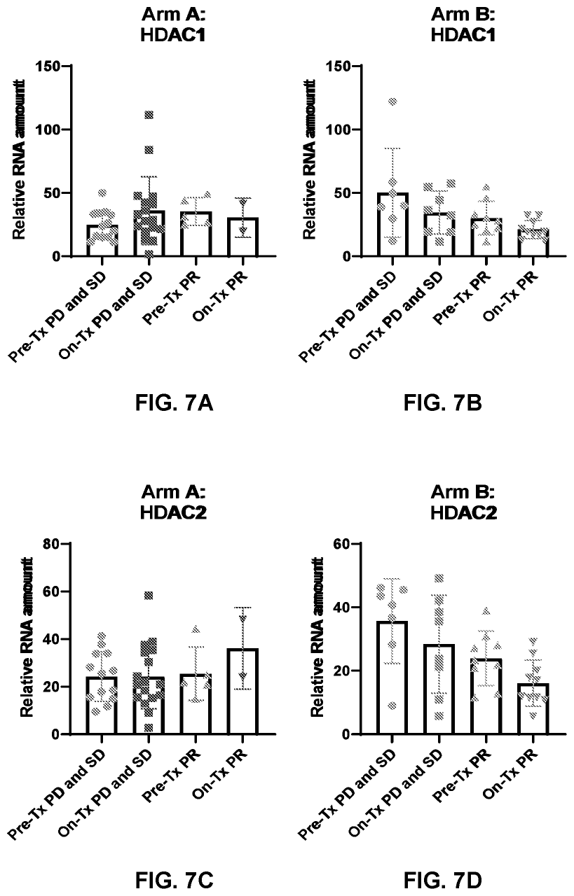

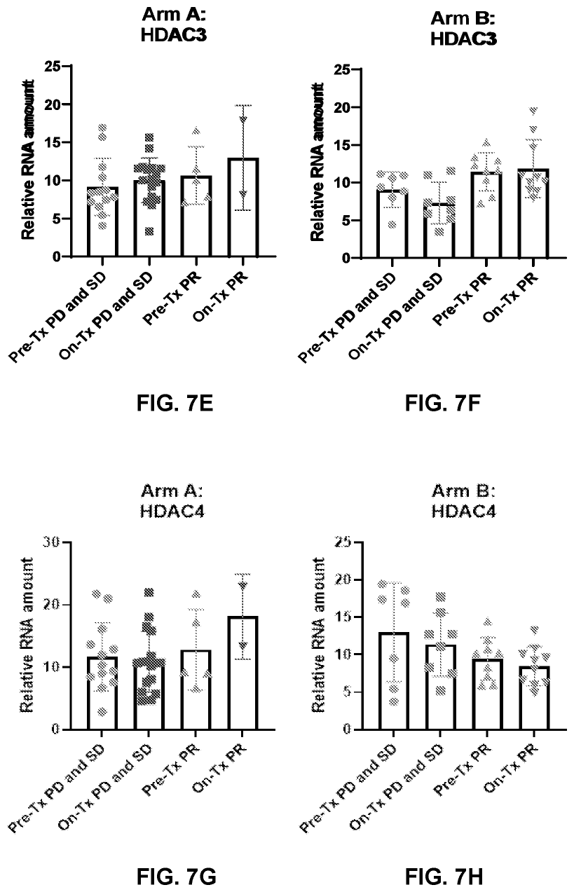

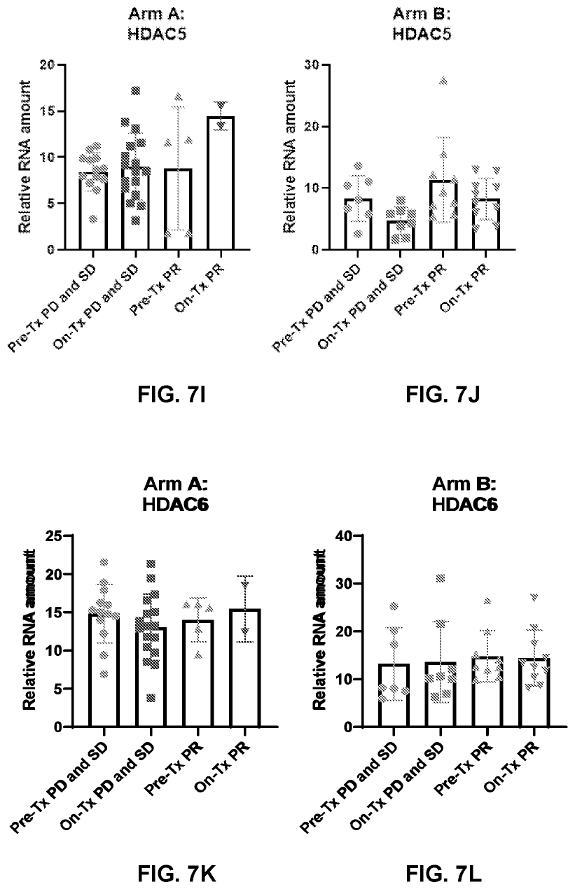

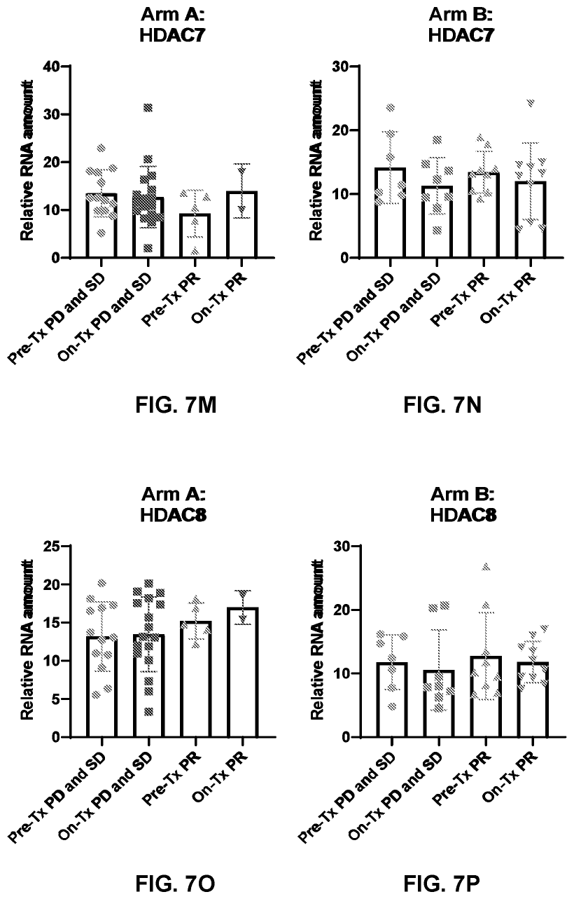

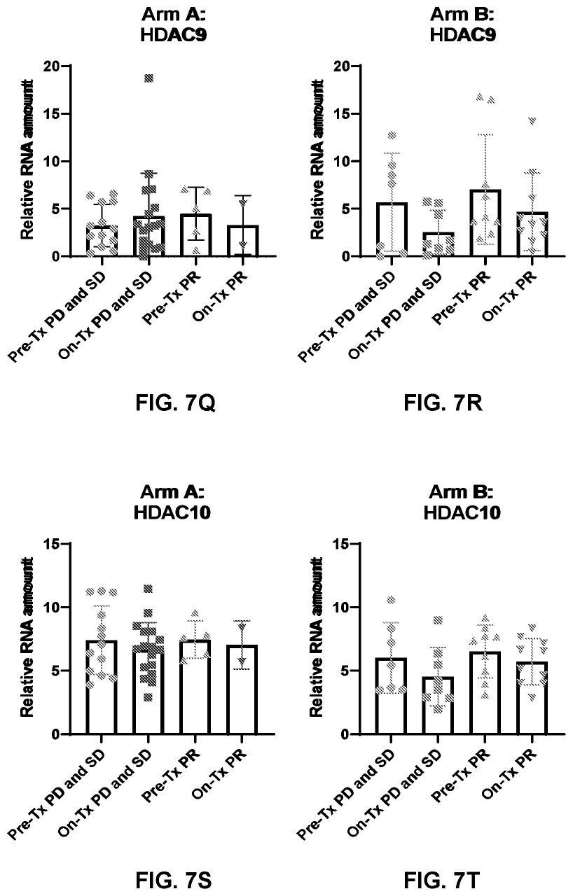

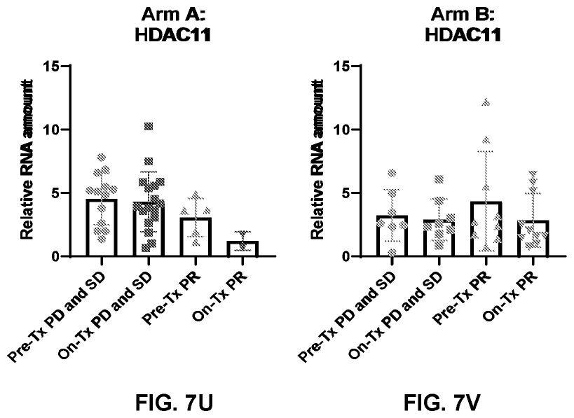

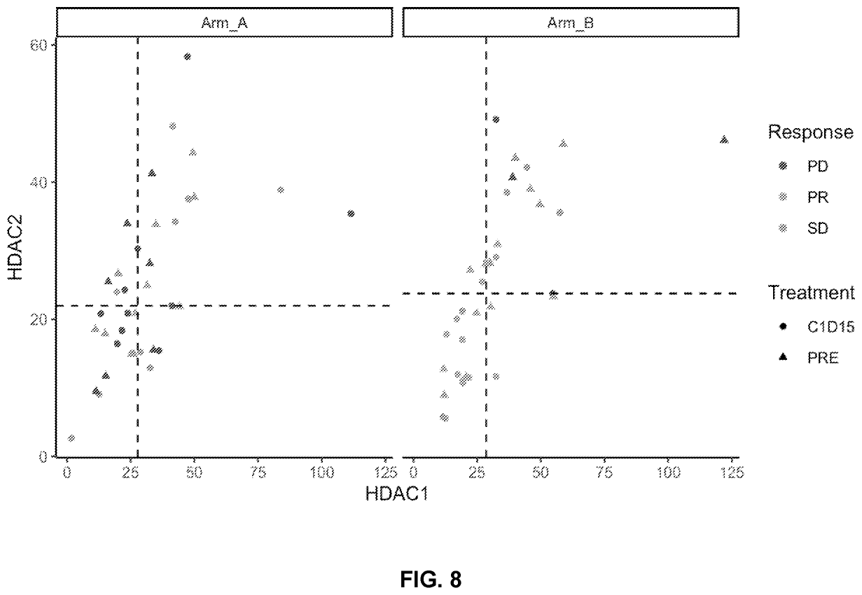

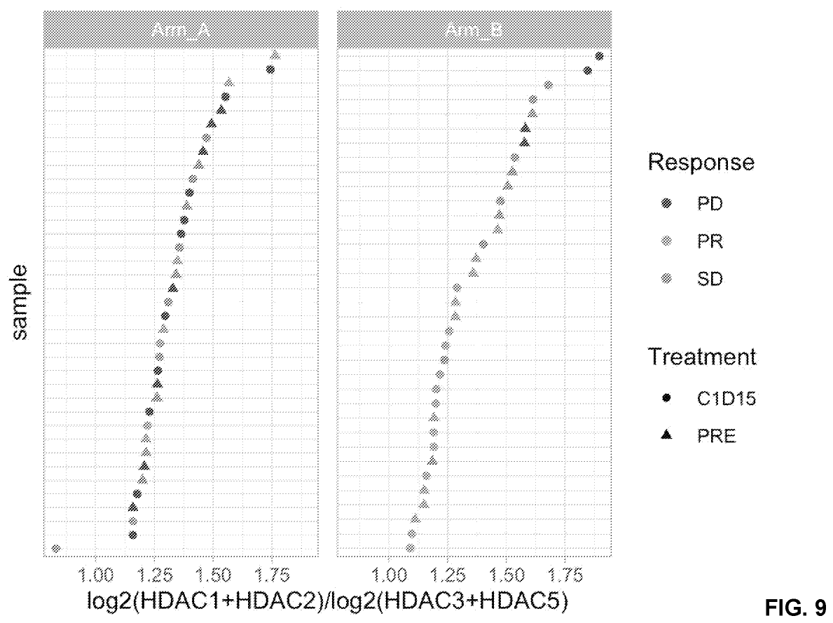

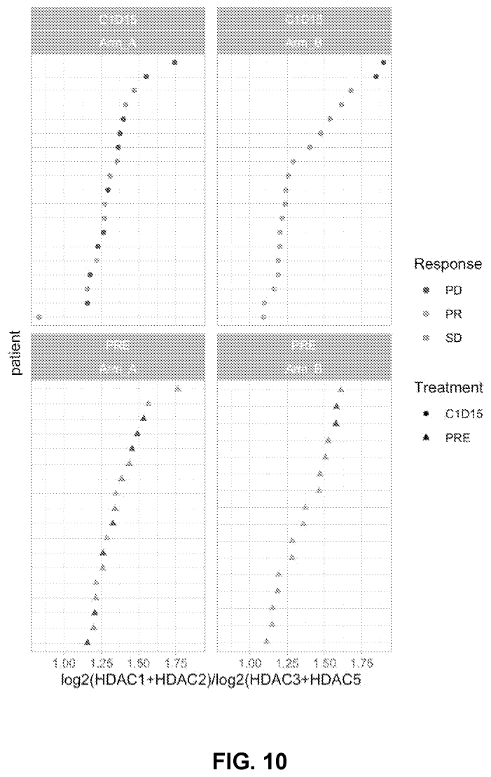

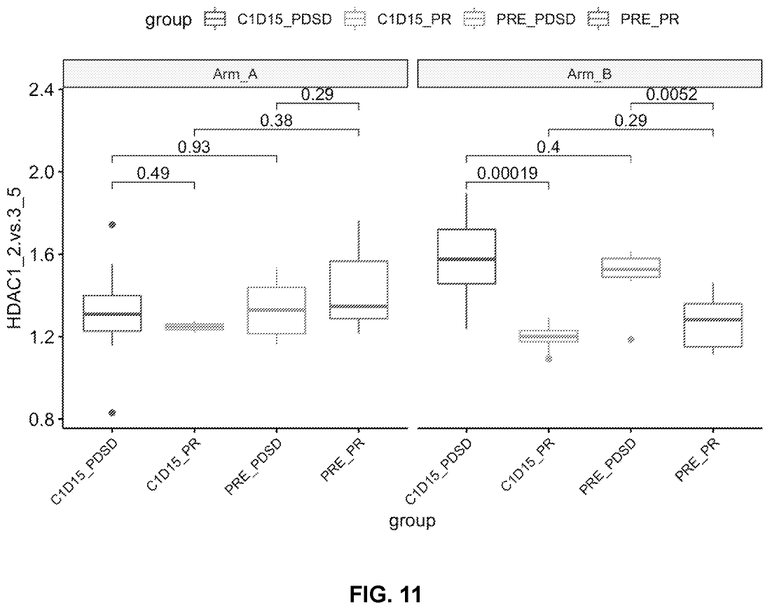

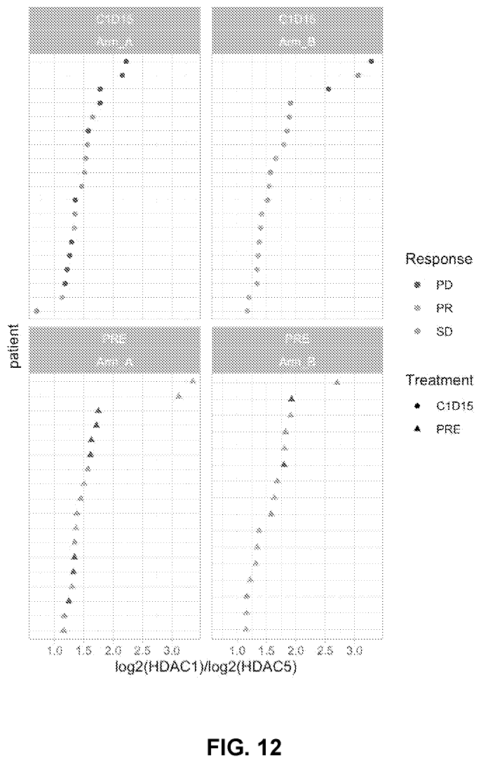

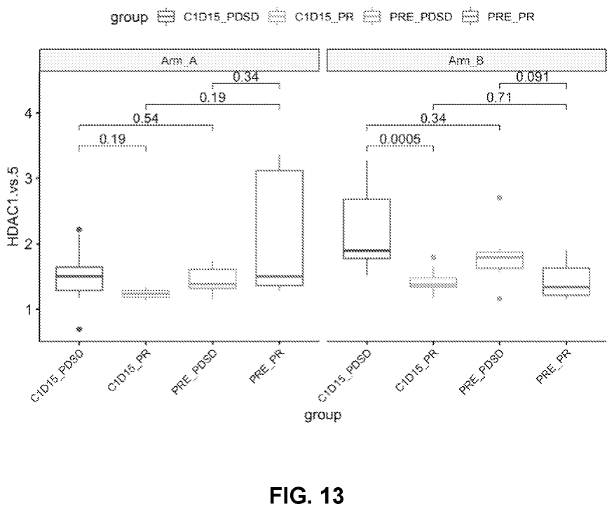

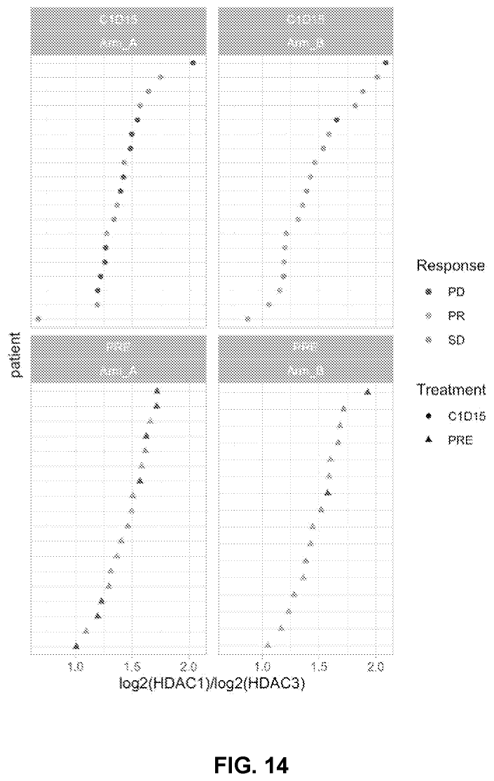

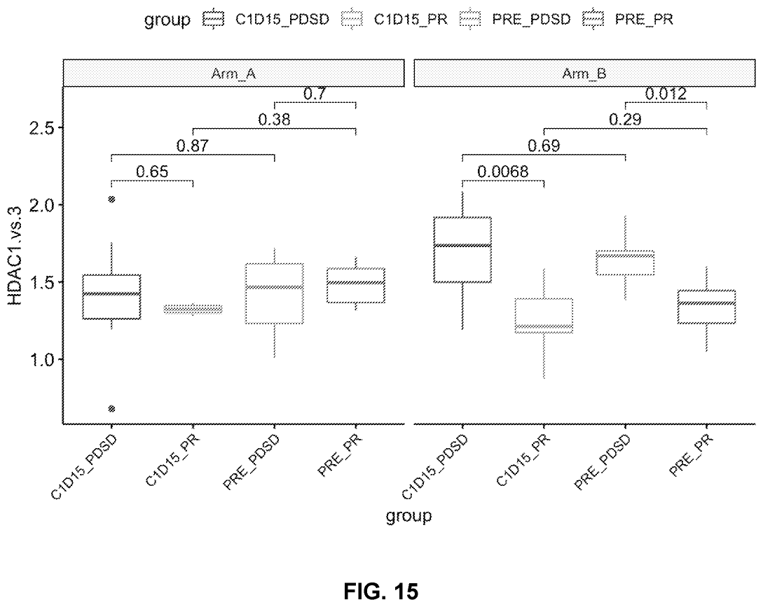

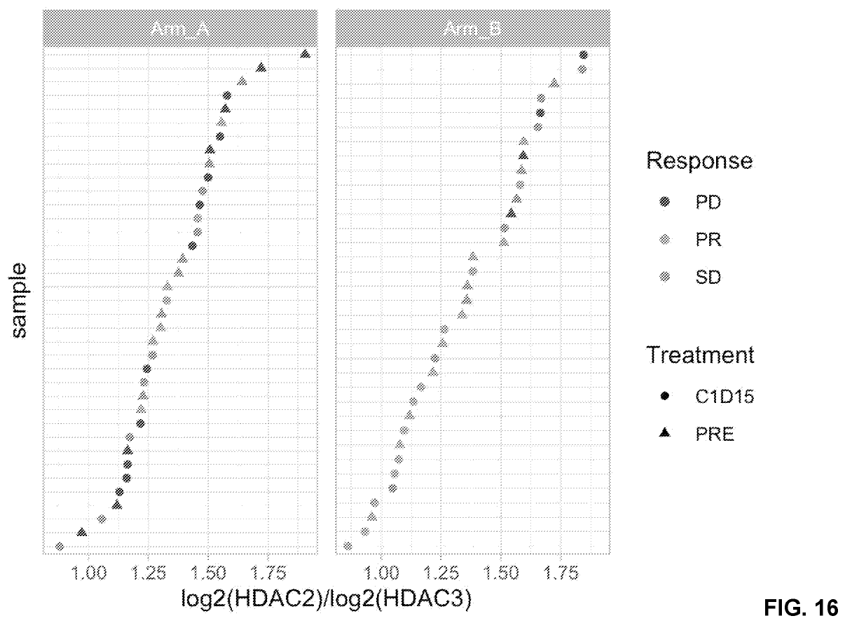

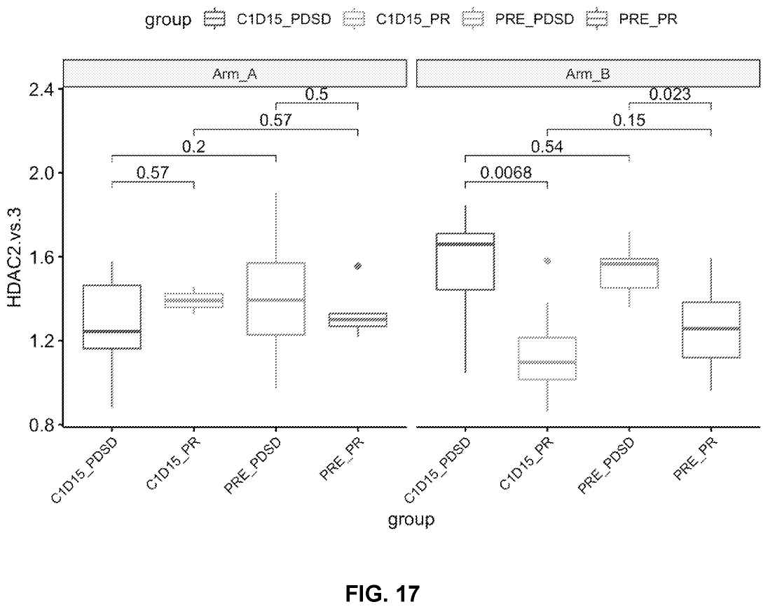

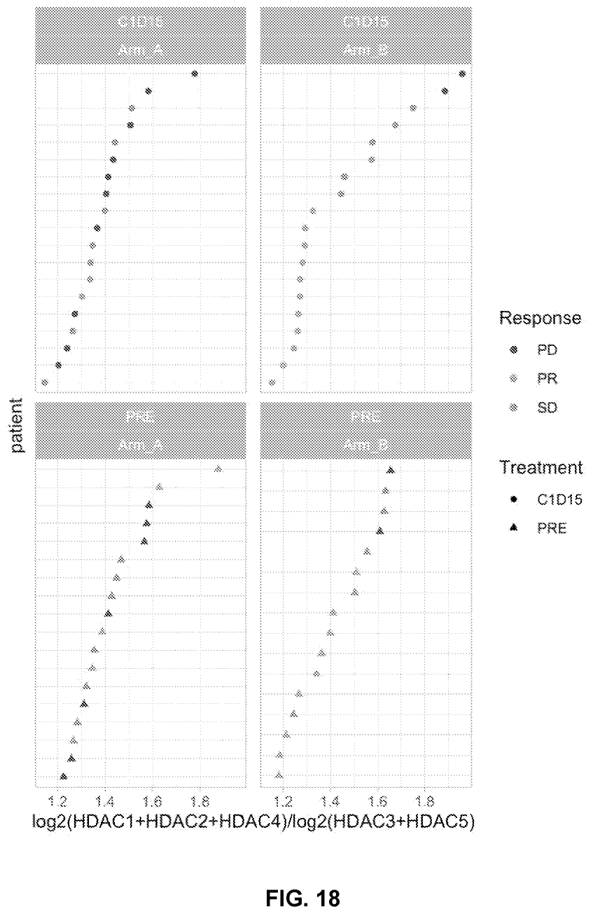

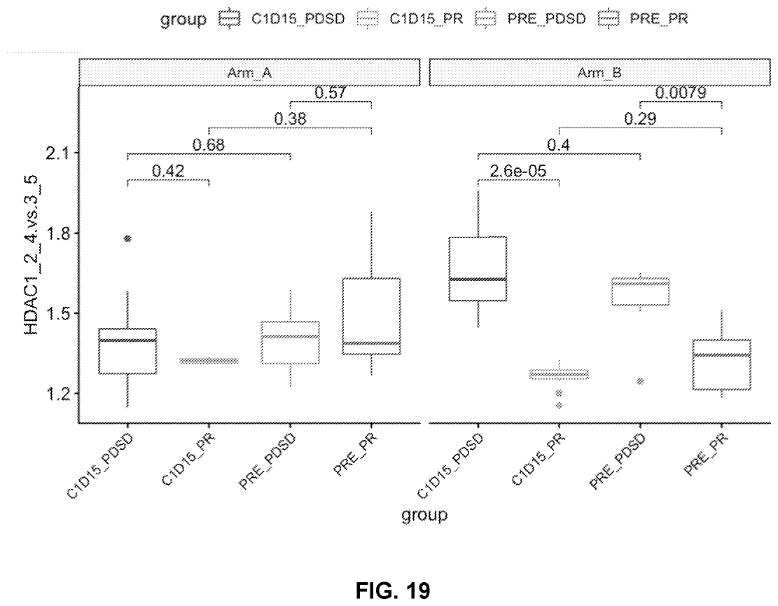

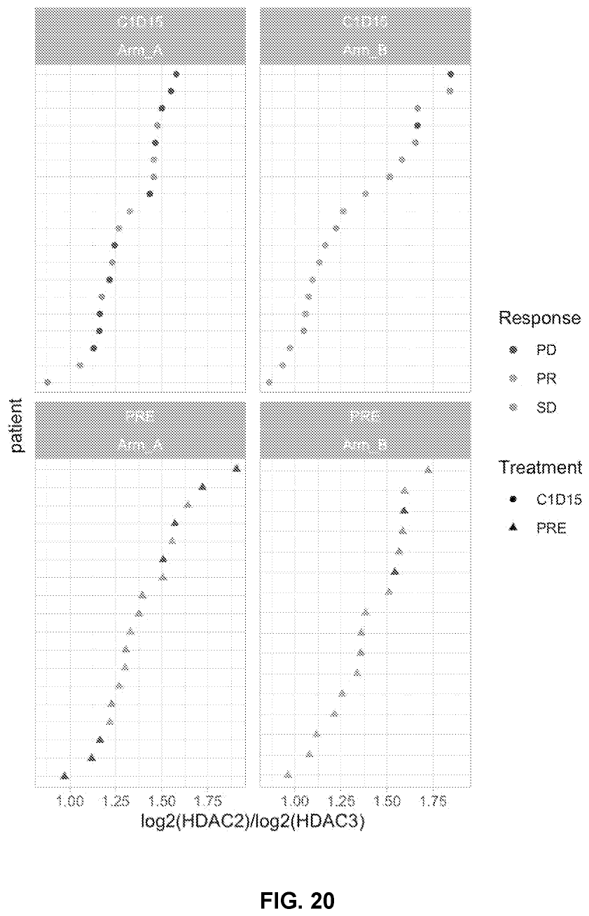

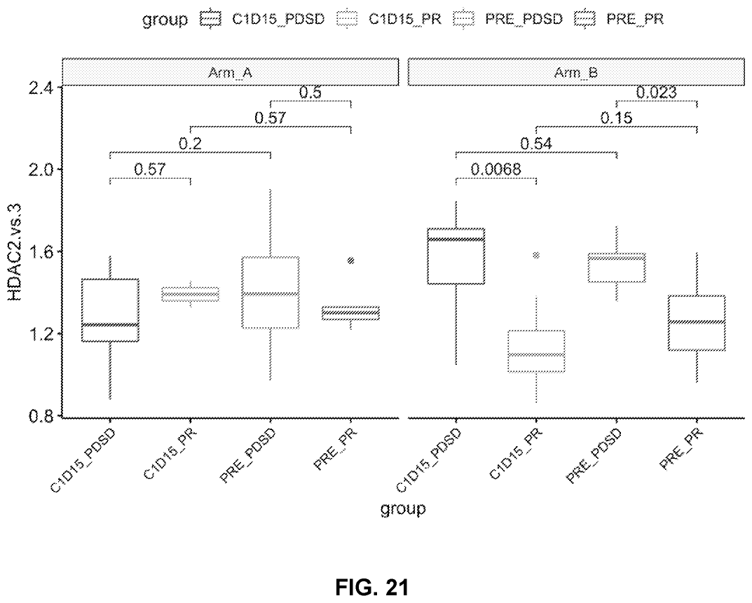

Relative RNA expression for HDAC1-11 was evaluated in Arm A and Arm B (FIGS. 6A to 6K). Relative RNA expression for HDAC1-11 was then evaluated in Arm A and Arm B for PD/SD vs. PR (FIGS. 7A to 7V), showing some trends. For example, Arm B PR biopsies had lower expression of HDAC1, 2 and 4, and higher expression of HDAC3 and 5 when compared to Arm B PD/SD biopsies. This was also evident in Pre-treatment biopsies. Arm A did not have a similar trend. FIG. 8 shows combined low expression of both HDAC1 and HDAC2 is associated with response. The ratios of HDAC expression was therefore evaluated. As shown in FIGS. 9-21, these ratios are predictive of treatment responsiveness. These are most clearly demonstrated in box plots (FIGS. 11, 13, 15, 17, 19, 21) showing statistically significant differences in HDAC ratios in Arm A but not Arm A. The ratios were also significantly different in Arm B pre-treatment biopsies (FIGS. 10, 20, 18) suggesting that pre-treatment biopsies can be sufficient to determine HDAC ratios associated with benefit.

### Example 3

### PD-1 Refractory NSCLC

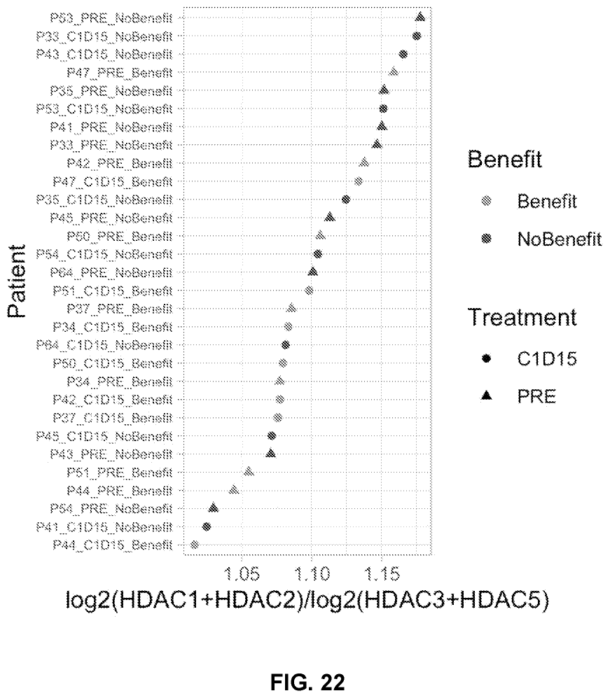

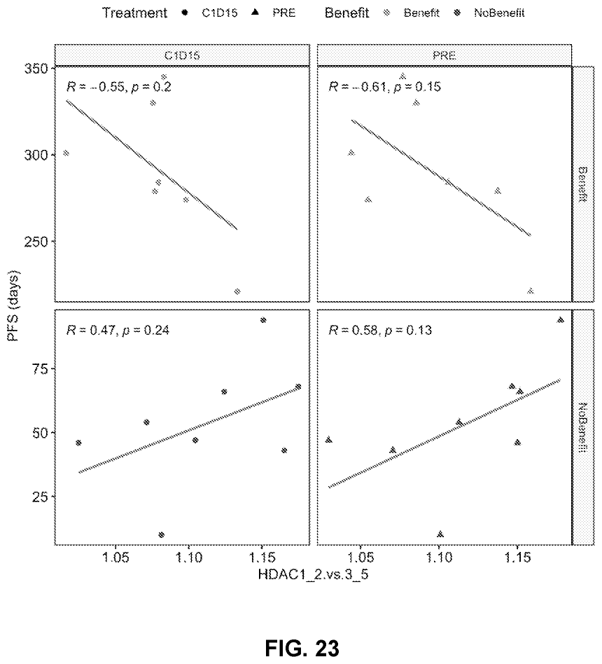

As shown in FIGS. 22 and 23, in immunotherapy naïve patients, low ratio of HDAC1+HDAC2/HDAC3+HDAC5 was strongly associated with ability of patients to respond to the combination pembrolizumab and HDAC inhibitor vorinostat treatment, but not pembrolizumab alone treatment.

A randomized, phase II trial was initiated for immunotherapy naïve patients with the primary objective to determine if the combination of pembrolizumab and HDAC inhibitor vorinostat had superior objective response rate (ORR) compared to pembrolizumab monotherapy. Interim analysis indicated that combination patients (Arm B, n=23) had 52% PRs compared to a 25% PRs in Arm A (n=24) patients (p=0.026) and a 91% disease control rate compared to 58% in Arm A (p=0.024). These results suggest that a biomarker that predicts patient benefit to anti-PD-1+HDAC inhibitor can be highly useful to select NSCLC patients for this combination treatment.

Gene expression data was obtained by RNA-sequencing (RNA-seq) of pre-treatment and early on-treatment (day 15-21 after treatment initiation) tumor biopsies from the above trial. These studies led to the identification of biomarker of response to the combination treatment based on expression of specific HDACs. Specifically, the ratio of HDAC1+HDAC2/HDAC3+HDAC5 was strongly associated with ability of patients to respond to the combination (p=0.0052) but not pembrolizumab alone treatment (p=0.29). This biomarker can be used for selecting patients who are likely to respond to anti-PD-1+HDACi combination therapy.

In a separate trial in anti-PD-1/PD-L1 refractory NSCLC patients, patients who derived anti-PD-1+HDAC inhibitor vorinostat combination treatment benefit (benefit defined as partial response or stable disease greater than 24 weeks) had lower ratio of HDAC1+HDAC2/HDAC3+HDAC5. Low ratio also had an associative trend with progression-free survival in patients who benefited from treatment but not those who did not benefit (Gray, J. E., et al. Clin Cancer Res. 2019 25(22):6623-6632).

### Example 4

### PD-1 Refractory Melanoma

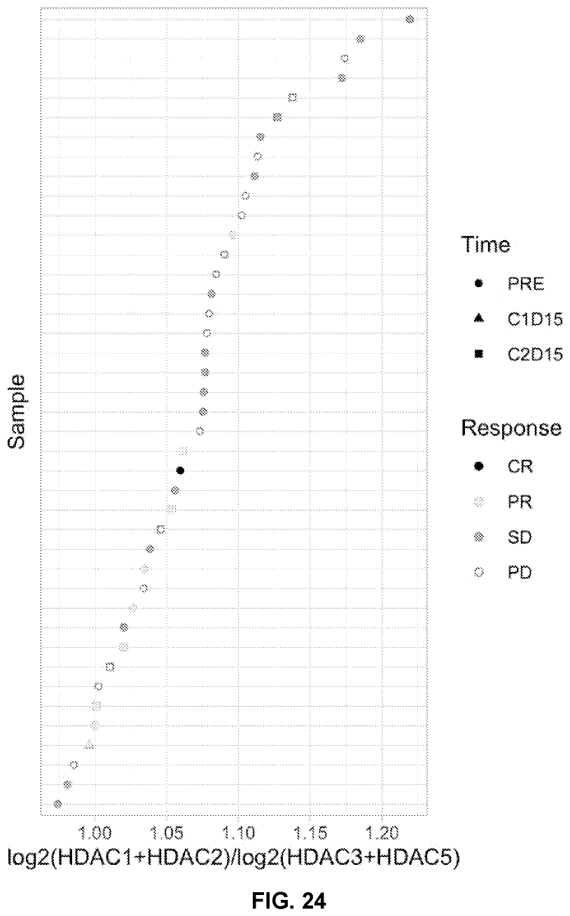

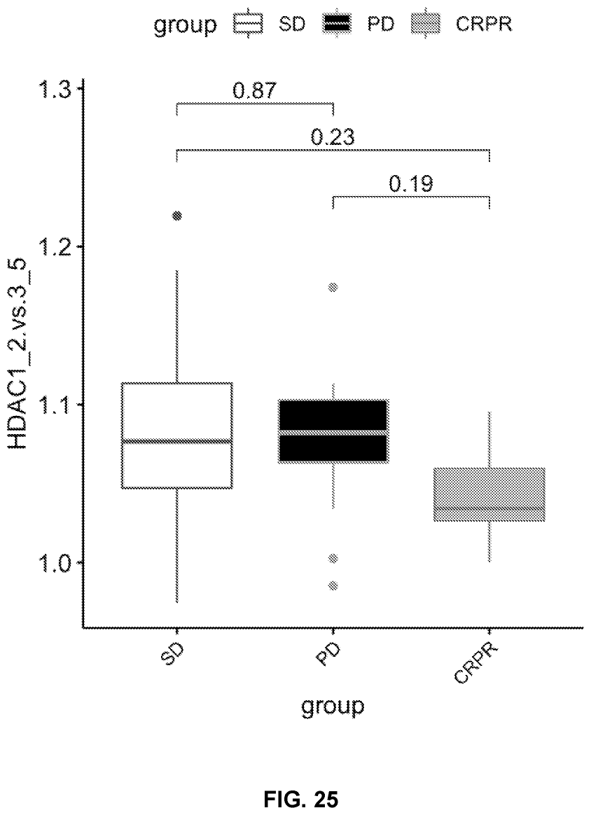

As shown in FIGS. 24 and 25, and Table 5 below, in anti-PD-1/PD-L1 refractory melanoma patients, low ratio of HDAC1+HDAC2/HDAC3+HDAC5 tended to be associated with complete or partial response to the combination of anti-PD-1+HDAC inhibitor entinostat.

TABLE 5
Melanoma

| | PRE | C1D15 | C2D15 |
|---|---|---|---|
| CR | 1 | 0 | 0 |
| PR | 4 | 1 | 4 |
| SD | 15 | 0 | 1 |
| PD | 12 | 0 | 3 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

2. A method for treating a solid tumor in a subject, comprising
(a) assaying a sample from the subject for expression of two or more Histone deacetylases (HDACs);
(b) determining a predicted response score from the expression of the two or more HDACs, wherein the response score predicts that the subject will respond and/or benefit from combination immunotherapy and HDAC inhibitor therapy; and
(c) administering to the subject a therapeutically effective amount of a combination of immunotherapy and an HDAC inhibitor.

3. The method of claim 1, wherein step (a) comprises assaying the sample for any combination of two or more of HDAC1, HDAC2, HDAC3, HDAC4, and HDAC5.

4. The method of claim 2, wherein step (a) comprises assaying the sample for HDAC1, HDAC2, HDAC3, HDAC4, and HDAC5.

5. The method of claim 2, wherein step (a) comprises assaying the sample for HDAC1, HDAC2, HDAC3, and HDAC5.

6. The method of claim 1, wherein step (b) comprises determining the ratio of a first set of HDACs to a second set of HDACs, wherein a ratio at or below 1.4 is an indication that the subject will respond to combination checkpoint inhibitor and HDACi therapy.

7. The method of claim 5, wherein the first set comprises HDAC1, HDAC2, and/or HDAC4 and the second set comprises HDAC3 and/or HDAC5.

8. The method of claim 6, wherein the first set comprises HDAC1 and HDAC2 and the second set comprises HDAC3 and HDAC5.

9. The method of claim 1, wherein step (a) further comprises assaying the sample for expression of a housekeeping gene, and wherein step (b) comprises normalizing expression of the two or more HDACs.

10. The method of claim 1, wherein the immunotherapy is a checkpoint inhibitor, vaccine, a monoclonal antibody, an oncolytic virus, a cell-based immunotherapy, or a radiopharmaceutical.

11. The method of claim 9, wherein the checkpoint inhibitor comprises an anti-PD-1 antibody, anti-PD-L1 antibody, anti-CTLA-4 antibody, or a combination thereof.

12. The method of claim 10, wherein the checkpoint inhibitor comprises pembrolizumab, nivolumab, cemiplimab, atezolizumab, avelumab, or durvalumab.

13. The method of claim 1, wherein the HDAC inhibitor is a class I HDAC inhibitor.

14. The method of claim 12, wherein the HDAC inhibitor is vorinostat, entinostat, romidepsin, or panabinostat.

15. The method of claim 12, wherein the HDAC inhibitor is HBI-8000.

16. The method of claim 1, wherein the tumor is a melanoma.

17. The method of claim 15, wherein the subject is refractory to immunotherapy.

18. The method of any claim 1, wherein the tumor is a non-small cell lung cancer (NSCLC)

19. The method of claim 17, wherein the subject is immunotherapy naïve.

20. The method of claim 1, further comprising treating the tumor cells with one or more additional therapeutic agents.
